(19) United States
(12) Patent Application Publication
(10) Pub. No.: US 2010/0204310 A1
(43) Pub. Date: Aug. 12, 2010

(54) NEW MODALITIES FOR TREATMENT OF DRUG-RESISTANT TUBERCULOSIS AND OTHER DISEASES

(75) Inventors: Paul C. Zamecnik, Boston, MA (US); Karen Pierson, Cambridge, MA (US); David Tabatadze, Worcester, MA (US)

Correspondence Address: PRETI, FLAHERTY, BELIVEAU & PACHIOS, LLP, PO Box 9546, One City Center, Portland, ME, 04112-9546, US

(73) Assignee: ZATA PHARMACEUTICALS, INC.

(21) Appl. No.: 12/556,933

(22) Filed: Sep. 10, 2009

Related U.S. Application Data
(60) Provisional application No. 61/096,382, filed on Sep. 12, 2008.

(51) Int. Cl. A61K 31/7088; C07H 21/02; A61P 31/06

(52) U.S. Cl. 514/44 R; 536/23.2

(57) ABSTRACT

The invention provides antibacterial compounds comprising an oligonucleotide having a sequence complementary to a translation initiation region of an mRNA encoding a mycolyl transferase of Mycobacterum tuberculosis selected from protein 30, 32A and 32B, and further having 5′ and 3′ palindromic hairpin-forming sequences, said compound being covalently linked to a protein synthesis inhibiting antibiotic via a linker. The invention further provides pharmaceutical formulations of such compounds and methods of use thereof for treating tuberculosis. Other diseases may similarly be treated by tethering and antibiotic which targets a ribosomal protein to an antisense oligonucleotide complementary to an mRNA involved in the disease.

### RELATED APPLICATIONS

This application claims the benefit of U.S. Provisional Patent Application Ser. No. 61/096,382, filed on Sep. 12, 2008, the entire contents of which are incorporated herein by reference.

### BACKGROUND OF THE INVENTION

1. Field of the Invention

The invention relates to the treatment of tuberculosis. More particularly, the invention relates to the treatment of infections by drug resistant Mycobacterium tuberculosis. The invention further relates to the treatment of other diseases by specific inhibition of both ribosome function and translation of specific mRNAs.

2. Summary of the Related Art

Tuberculosis, one of the leading causes of death worldwide, is caused primarily by the facultative intracellular bacterium Mycobacterium tuberculosis. Over the past several years, many strains of M. tuberculosis have developed resistance to antibiotics. One cause of this resistance is the formation of a extra-cellular wall structure that makes it difficult for antibiotics to penetrate the cell wall.

Belisle et al, Science 276: 1420-1422 (1997), teaches that M. tuberculosis expresses and secretes three closely related mycolyl transferases that catalyze the transfer of mycolic acid from one trehalose 6-monomycolate molecule to another, resulting in the formation of free trehalsose and trehalose 6,6′-dimycolate, which is subsequently incorporated into the cell wall. Cole et al, Nature 393: 537-544 (1998), teaches that these three mycolyl transferases, proteins 32A, 30 and 32B are encoded by three different, unlinked single operon genes, called fbpA, fbpB and fbpC.

These mycolyl transferase genes have gathered interest as potential antibacterial targets. Harth et al., Proc. Natl. Acad. Sci. USA 104: 7199-7204 (2007) teaches that antisense oligonucleotides complementary to the translation initiation sites for the transcripts of these there genes and having 5′ and 3′ hairpin extensions, inhibit growth of M. tuberculosis in broth cultures and in human macrophages.

Given the importance of developing treatments for drug resistant tuberculosis, new modalities for increasing the effectiveness of such treatments are needed.

Treatment of other diseases, such as viral, other bacterial, fungal and protozal or other parasitic infections, as well as cancer and genetic or metabolic aberrations may benefit from such modalities as well.

### BRIEF SUMMARY OF THE INVENTION

The present invention provides new modalities for treating drug resistant tuberculosis. This invention exploits the facts that antisense oligonucleotides readily penetrate the M. tuberculosis cell wall, and in modified form can inhibit bacterial growth by inhibiting expression of the three mycolyl tranferases. The present inventors have surprisingly discovered that such modified antisense oligonucleotides can provide enhanced antibacterial effects by acting as carriers of protein synthesis inhibiting antibiotics and delivering them to the mRNA transcript where they interact with the ribosome to block synthesis of the mycolyl transferase proteins.

In a first aspect, the invention provides novel antibacterial compounds comprising antisense oligonucleotides complementary to the translation initiation region of mRNAs encoding M. tuberculosis 32A, 30 and 32B proteins, having 5′ and 3′ hairpin-forming extensions, and covalently linked to a 70S ribosome protein synthesis inhibitor antibiotic via a linker.

In a second aspect, the invention provides pharmaceutical formulations comprising an antibacterial compound according to the invention and a pharmaceutically acceptable diluents, salt, excipient or carrier.

In a third aspect, the invention provides methods for treating drug resistant tuberculosis comprising administering to a mammal infected with drug resistant M. tuberculosis an antibacterial compound or pharmaceutical formulation according to the invention.

In a fourth aspect, the invention provides compositions and methods for treating other diseases, such as viral, other bacterial, fungal and protozal or other parasitic infections, as well as cancer and genetic or metabolic aberrations through the use of an antibiotic specific for a ribosomal protein tethered to an antisense oligonucleotide complementary to an mRNA involved in the pathology of the disease.

### DETAILED DESCRIPTION OF THE PREFERRED EMBODIMENTS

The invention relates to the treatment of tuberculosis. More particularly, the invention relates to the treatment of infections by drug resistant Mycobacterium tuberculosis. The references cited herein reflect the knowledge in the art and are hereby incorporated by reference in their entirety. Any conflicts between the cited references and this specification shall be resolved in favor of the latter.

The present invention provides new modalities for treating drug resistant tuberculosis. This invention exploits the facts that antisense oligonucleotides readily penetrate the M. tuberculosis cell wall, and in modified form can inhibit bacterial growth by inhibiting expression of the three mycolyl tranferases. The present inventors have surprisingly discovered that such modified antisense oligonucleotides can provide enhanced antibacterial effects by acting as carriers of protein synthesis inhibiting antibiotics and delivering them to the mRNA transcript where they interact with the ribosome to block synthesis of the mycolyl transferase proteins.

In a first aspect, the invention provides novel antibacterial compounds comprising antisense oligonucleotides complementary to the translation initiation sites of mRNAs encoding M. tuberculosis 32A, 30 and/or 32B proteins, having 5′ and 3′ hairpin-forming extensions, and covalently linked to a 70S ribosome protein synthesis inhibitor antibiotic via a linker.

According to this aspect of the invention, antibiotics are attached via a tether, or linker, to an oligonucleotide at one or more of a variety of positions. Preferred positions include the 3′ end, 5′ end, on the heterocyclic bases and on the internucleoside linkages.

The tether, or linker, has an anchor group for such attachment, which may preferably selected from carboxyl, carbonyl, hydroxyl, amino, phosphate, thiophosphate and the like. The linker is preferably from about 2 to about 50 atomic bond lengths in size. The linker may be of any suitable chemistry, including alkyl, ether, amido, or other hydrophobic, hydrophilic or amphipathic moieties. The antibiotic may be attached to the linker through chemically active groups on the antibiotic. Such covalent linkage may be physiologically irreversible, or slowly reversible under physiological conditions.

Preferred antibiotics include, without limitation, streptomycin, Sordarin, viomycin, ciprofloxacin, spectinomycin, thiostrepton, virginiamycin M, spramycin I-III, tylosin, carbomycin A, azithromycin, troleandomycin, centhromycin, NRI-A72310, oxazolidinones, lincosamides, sparsomysin, blasticidin S, anisomycin, puromycin, chloramphenicol, tRNA-A76, pulvomycin, enacycloxin IIa, kirromycin, aminoglycosides, tetracycline, glycylcyclines, avilamycin, kasugamycin, pactamycin, edeine and cancer chemotherapeutics.

The oligonucleotide comprises a nucleotide sequence complementary to the translation initiation region of an mRNA encoding mycolyl tranferase protein 30, 32A or 32B. It further comprises a palindromic, hairpin-forming sequence at its 5′ and 3′ ends. The term “complementary to the translation initiation region of an mRNA” means sufficiently complementary to hybridize via Watson-Crick base pairing under physiological conditions to a sequence of the mRNA that includes the translation initiation codon. The length of the nucleotide sequence complementary to the translation initiation region is preferably from about 15 to about 50 nucleotides, more preferably from about 15 to about 35 nucleotides.

Non-limiting examples of oligonucleotides according to the invention are as follows:

30:
5′-GCGCATATGCGaatctttcggctcacgtctgtcatGCGCGCGC-3′
32A:
5′-GCGCATATGCGacgaaccctgtcaacaagctgcatGCGCGCGC-3′
32B:
5′-GCGCATATGCGtcgcacctgttcgaagaacgtcatGCGCGCGC-3′.

wherein upper case indicates palindromic hairpin-forming sequence and lower case indicates sequence complementary to translation initiation site.

For purposes of the invention, the term “oligonucleotide” refers to a polynucleoside formed from a plurality of linked nucleoside units. Such oligonucleotides can be obtained from existing nucleic acid sources, including genomic or cDNA, but are preferably produced by synthetic methods. In some embodiments each nucleoside unit includes a heterocyclic base and a pentofuranosyl, 2′-deoxypentfuranosyl, trehalose, arabinose, 2′-deoxy-2′-substituted arabinose, 2′-O-substituted arabinose or hexose sugar group. The nucleoside residues can be coupled to each other by any of the numerous known internucleoside linkages. Such internucleoside linkages include, without limitation, phosphodiester, phosphorothioate, phosphorodithioate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. The term “oligonucleotide” also encompasses polynucleosides having one or more stereospecific internucleoside linkage (e.g., (RP)- or (SP)-phosphorothioate, alkylphosphonate, or phosphotriester linkages). As used herein, the term “oligonucleotide” is expressly intended to include polynucleosides having any such internucleoside linkage, whether or not the linkage comprises a phosphate group. In certain embodiments, these internucleoside linkages may be phosphodiester, phosphorothioate, or phosphorodithioate linkages, or combinations thereof.

The term “oligonucleotide” also encompasses polynucleosides having additional substituents including, without limitation, protein groups, lipophilic groups, intercalating agents, diamines, folic acid, cholesterol and adamantane. The term “oligonucleotide” also encompasses any other nucleobase containing polymer, including, without limitation, peptide nucleic acids (PNA), peptide nucleic acids with phosphate groups (PHONA), locked nucleic acids (LNA), morpholino-backbone oligonucleotides, and oligonucleotides having backbone sections with alkyl linkers or amino linkers.

The oligonucleotides of the invention can include naturally occurring nucleosides, modified nucleosides, or mixtures thereof. As used herein, the term “modified nucleoside” is a nucleoside that includes a modified heterocyclic base, a modified sugar moiety, or a combination thereof. In some embodiments, the modified nucleoside is a non-natural pyrimidine or purine nucleoside, as herein described. In some embodiments, the modified nucleoside is a 2′-substituted ribonucleoside an arabinonucleoside or a 2′-deoxy-2′-substituted-arabinoside.

For purposes of the invention, the term “2′-substituted ribonucleoside” or “2′-substituted arabinoside” includes ribonucleosides or arabinonucleosides in which the hydroxyl group at the 2′ position of the pentose moiety is substituted to produce a 2′-substituted or 2′-O-substituted ribonucleoside. In certain embodiments, such substitution is with a lower alkyl group containing 1-6 saturated or unsaturated carbon atoms, or with an aryl group having 6-10 carbon atoms, wherein such alkyl, or aryl group may be unsubstituted or may be substituted, e.g., with halo, hydroxy, trifluoromethyl, cyano, nitro, acyl, acyloxy, alkoxy, carboxyl, carboalkoxy, or amino groups. Examples of 2′-O-substituted ribonucleosides or 2′-O-substituted-arabinosides include, without limitation 2′-O-methylribonucleosides or 2′-O-methylarabinosides and 2′-O-methoxyethoxyribonucleosides or 2′-O-methoxyethoxyarabinosides.

The term “2′-substituted ribonucleoside” or “2′-substituted arabinoside” also includes ribonucleosides or arabinonucleosides in which the 2′-hydroxyl group is replaced with a lower alkyl group containing 1-6 saturated or unsaturated carbon atoms, or with an amino or halo group. Examples of such 2′-substituted ribonucleosides or 2′-substituted arabinosides include, without limitation, 2′-amino, 2′-fluoro, 2′-allyl, and 2′-propargyl ribonucleosides or arabinosides.

The term “oligonucleotide” includes hybrid and chimeric oligonucleotides. A “chimeric oligonucleotide” is an oligonucleotide having more than one type of internucleoside linkage. One non-limiting example of such a chimeric oligonucleotide is a chimeric oligonucleotide comprising a phosphorothioate, phosphodiester or phosphorodithioate region and non-ionic linkages such as alkylphosphonate or alkylphosphonothioate linkages (see e.g., Pederson et al. U.S. Pat. Nos. 5,635,377 and 5,366,878).

A “hybrid oligonucleotide” is an oligonucleotide having more than one type of nucleoside. One non-limiting example of such a hybrid oligonucleotide comprises a ribonucleotide or 2′ substituted ribonucleotide region, and a deoxyribonucleotide region (see, e.g., Metelev and Agrawal, U.S. Pat. Nos. 5,652,355, 6,346,614 and 6,143,881).

The term “compound” or “compound according to the invention” is intended to include N-oxides, hydrates, solvates, pharmaceutically acceptable salts, prodrugs and complexes thereof, and racemic and scalemic mixtures, diastereomers, enantiomers and tautomers thereof.

The compounds of the present invention may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g. methyl, ethyl, propyl and butyl chlorides, bromides and iodides), dialkyl sulfates (e.g. dimethyl, diethyl, dibuty and diamyl sulfates), long chain halides (e.g. decyl, lauryl, myristyl and stearyl chlorides, bromides and iodides), aralkyl halides (e.g. benzyl and phenethyl bromides), and others.

As used herein, the term “pharmaceutically acceptable salts” is intended to mean salts that retain the desired biological activity of the above-identified compounds and exhibit minimal or no undesired toxicological effects.

In a second aspect, the invention provides pharmaceutical formulations comprising an antibacterial compound according to the invention and a pharmaceutically acceptable carrier, excipient, or diluent. Compositions of the invention may be formulated by any method well known in the art and may be prepared for administration by any route, including, without limitation, parenteral, oral, sublingual, transdermal, topical, intranasal, intratracheal, or intrarectal. In certain preferred embodiments, compositions of the invention are administered intravenously in a hospital setting. In certain other preferred embodiments, administration may preferably be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term “pharmaceutically acceptable salt(s)” refers to salts that retain the desired biological activity of the above-identified compounds and exhibit minimal or no undesired toxicological effects. Examples of such salts include, but are not limited to, salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, palmoic acid, alginic acid, polyglutamic acid, naphthalenesulfonic acid, naphthalenedisulfonic acid, methanesulfonic acid, p-toluenesulfonic acid and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

In a third aspect, the invention provides methods for treating drug resistant tuberculosis comprising administering to a mammal infected with drug resistant M. tuberculosis an antibacterial compound or pharmaceutical formulation according to the invention. In certain preferred embodiments, compounds having complementarity to the translation initiation regions any one of the three mycolyl transferase mRNAs, 30, 32A and 30B are administered to a patient. In certain preferred embodiments, compounds having complementarity to the translation initiation regions of all three mycolyl transferase mRNAs, 30, 32A and 30B are administered together.

In a fourth aspect, the invention provides compositions and methods for treating other diseases, such as viral, other bacterial, fungal and protozal or other parasitic infections, as well as cancer and genetic or metabolic aberrations through the use of an antibiotic specific for a ribosomal protein tethered to an antisense oligonucleotide complementary to a translation start site of an mRNA involved in the pathology of the disease.

For example, an antibiotic that targets a ribosomal protein my be tethered to an antisense oligonucleotide complementary to the translation start site of HIV-I gag protein mRNA, the 3′ end of influenza virus C PBI gene viral RNA, c-myb mRNA, c-myc mRNA, Huntington's Disease mutant protein mRNA, and the mRNA encoding dihydrofolate reductase or ribonucleotide reductase of Plasmodium falciparum.

Administration of the compounds or pharmaceutical compositions according to the invention may be by any pharmaceutically acceptable route, including without limitation, parenteral, oral and intranasal administration.

The following examples are intended to further illustrate certain preferred embodiments of the invention and are not intended to limit the scope of the invention.

### EXAMPLE 1

### Attachment of Streptomycin to an Internucleotide Thiophosphate

Attachment of streptomycin through an internucleotide thiphosphate is shown in Scheme 1. After reaction between primary amino group of oligo (I) and aldehyde group of Streptomycin (II) a Schiff base bond is formed. Reduction with NaBH₄ can be used, if desirable, to strengthen covalent attachment of the oligomer to Streptomycin (IV).

As shown on schemes 1-4 below, streptomycin was attached to the oligonucleotide through amino linker at basic pH and Schiff base was reduced by NaBH₄.

In schemes 1-4 are demonstrated structurally different conjugates of an oligonuleotide to the streptomycin which could be divided into two groups: a chemically stable one, and one slowly degradable in a physiological environment. Prior to reduction of Schiff bases (components III, II, II and II in schemes 1-4 accordingly could be reversible in conditions close to physiological, and therefore the oligonucleotide could serve as a carrier of antibiotic molecules at the genetically designated target. After attachment of antibiotic to the oligonuclotide, these components can serve as double inhibitors of the transcription/translation site of the desirable genetic target. Oligonucleotides themselves serve as inhibitors of genetic activity of targeted genes. Antibiotics covalently attached to the oligonucleotides serve at a minimum as an extra steric blocker for inhibition of functioning of targeted gene, and/or as a modifier of ribosomal units participating in the translation process. More then one antibiotic molecule can be attached to one molecule of oligonucleotide, at different locations. Another example of an antibiotic containing an aldehyde group and which could be attached to the oliginucleotide like streptomycin is Sordarin (GM 193663). Antibiotics with amino groups also can be attached to the anchor group of linker covalently attached to the oligonucleotide. In the case of an amino group on an antibiotic, an anchor group on oligonucleotide can be phosphate or carboxyl. An example of antibiotic epsilon amino group is viomycin.

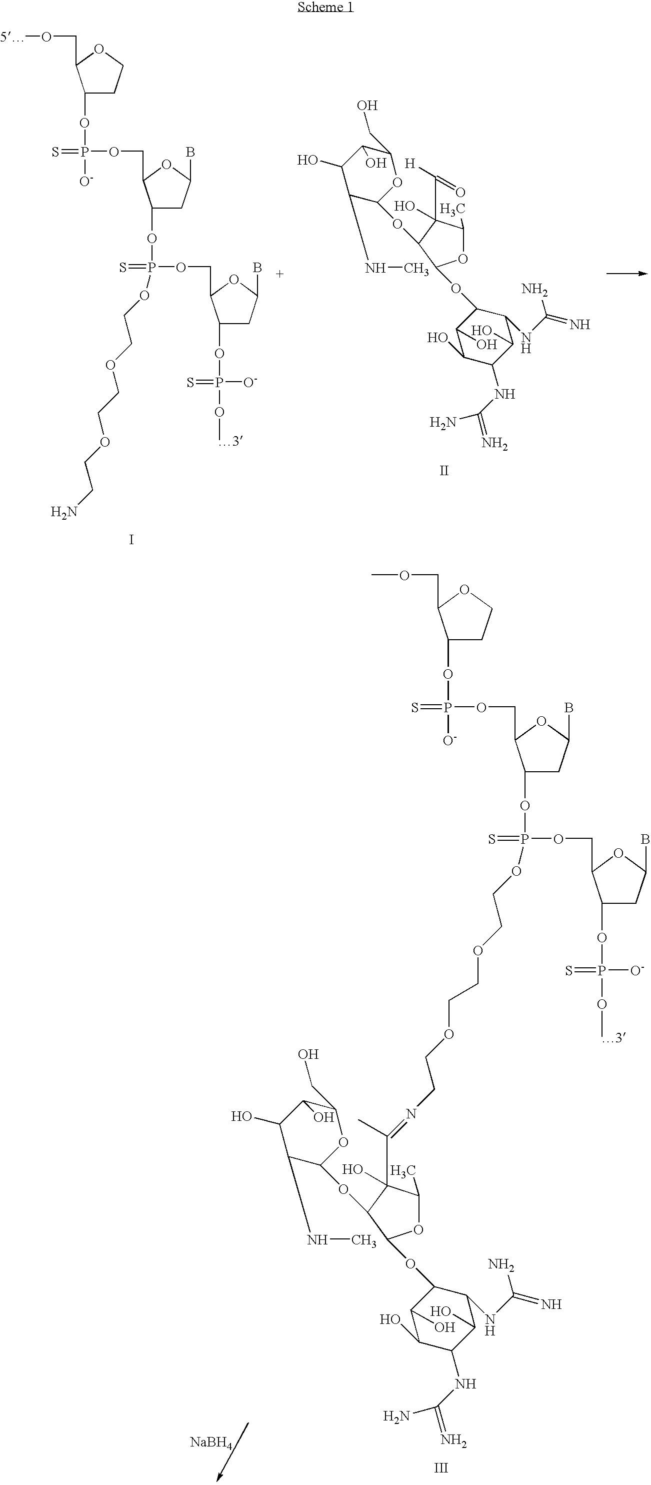

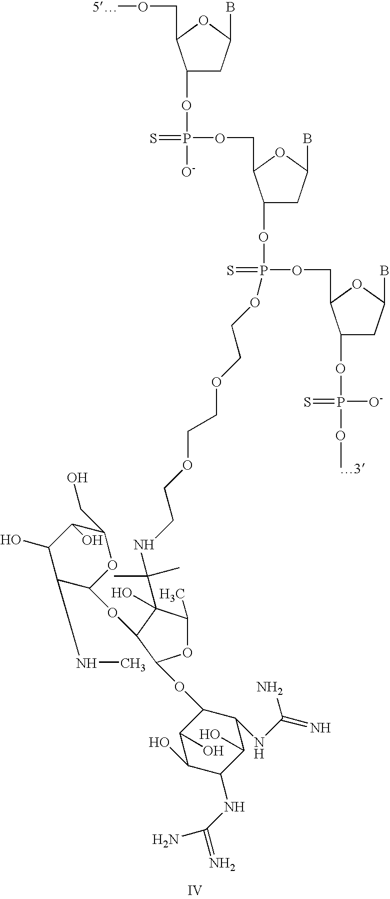

### EXAMPLE 2

### Attachment of Streptomycin to an Oligonucleotide at the 5′ End

Attachment of streptomycin to an oligonucleotide at the 5′ end is shown in Scheme 2. After reaction between 5′ primary amino group to streptomycin conjugate through a Schiff base bond (I) is formed, which later was reduced with NaBH₄ and a firm conjugate of oligo to streptomycin (II) formed.

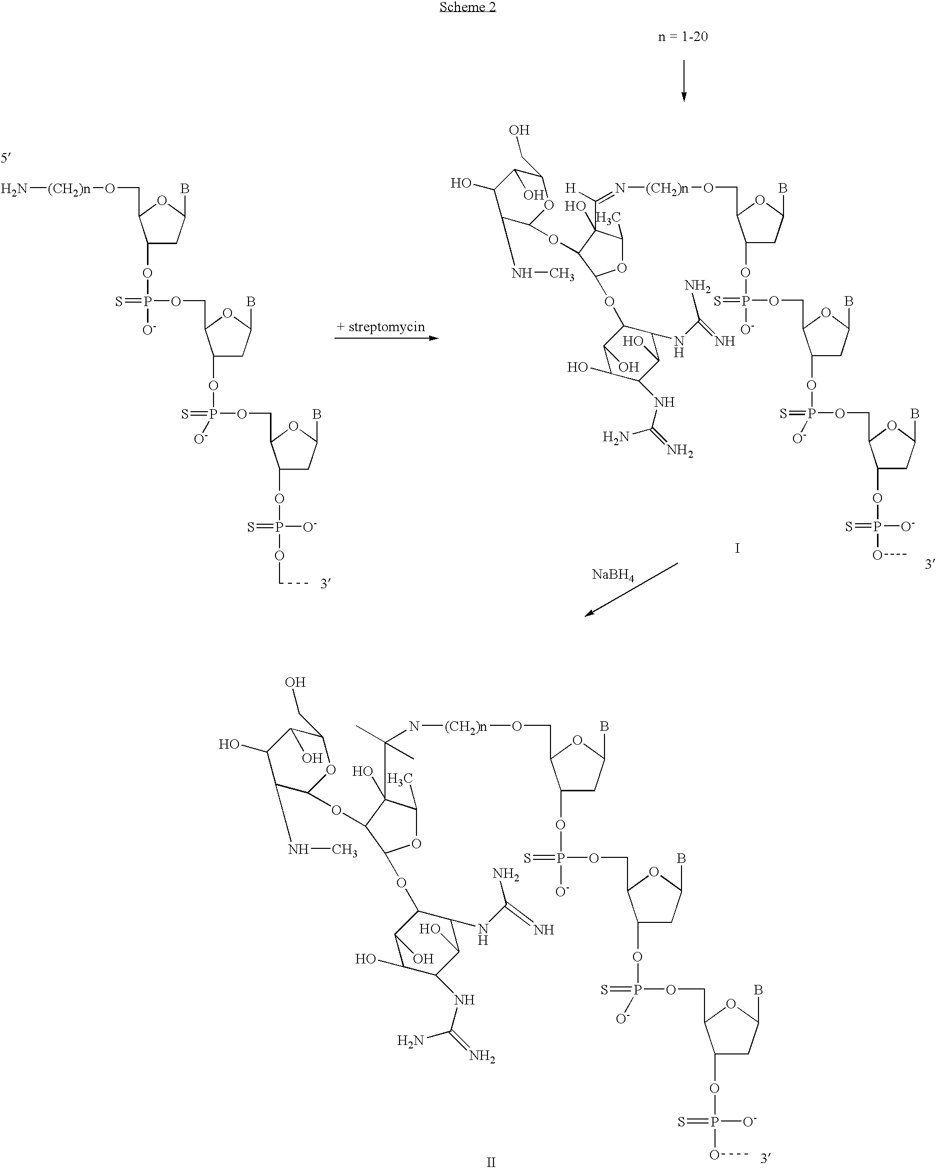

### EXAMPLE 3

### Attachment of Streptomycin to an Oligonucleotide at the 3′ End

Attachment of streptomycin to an oligonucleotide at the 3′ end is shown in Scheme 3. Reduction of Streptomycin conjugate to the oligo through a Schiff base bond (I) was done with NaBH₄ and firm conjugate of oligomer to streptomycin (II) was formed. Conjugate of streptomycin to the oligomer through a Schiff base bond (I) was formed as is shown in scheme 2.

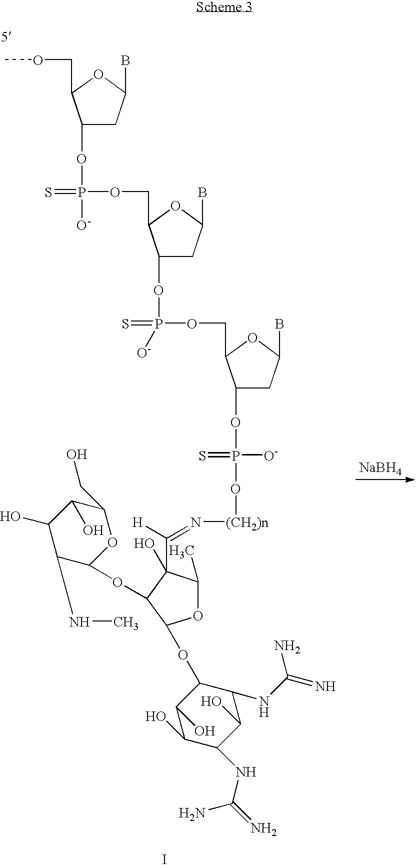

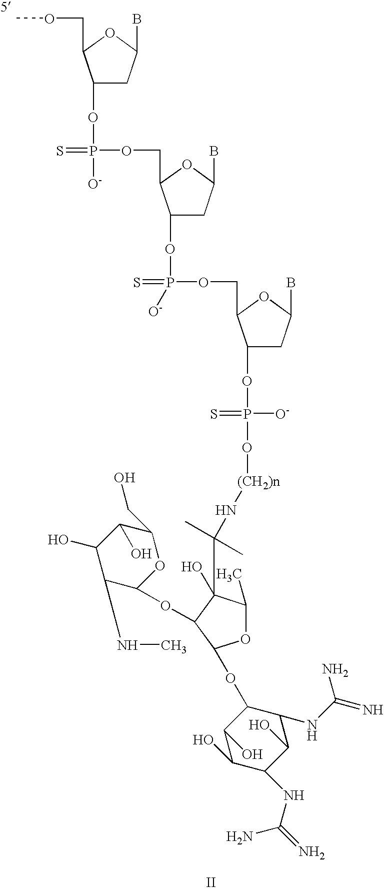

### EXAMPLE 4

### Attachment of Streptomycin to an Oligonucleotide at a Heterocyclic Base

Attachment of streptomycin to an oligonucleotide at a heterocyclic base is shown in Scheme 4. Reduction of Streptomycin conjugate to the oligomer through a Schiff base bond (I) was with NaBH₄ and formation of conjugate of oligomer to streptomycin (II). B stands for heterocycle base.

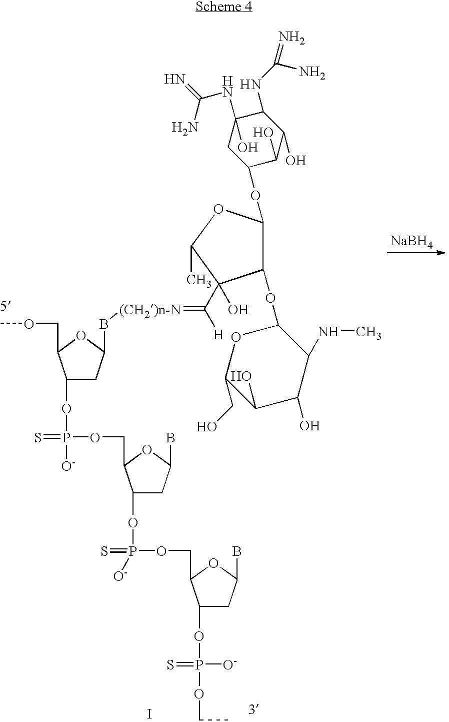

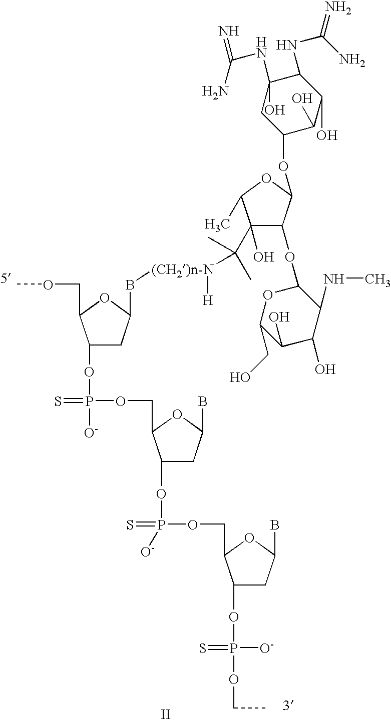

### EXAMPLE 5

### Different Types of Covalent Bonds Between an Antibiotic and an Oligonucleotide

Different types of covalent bonds between an antibiotic and an oligonucleotide are shown in Scheme 5. When X represents an oligonucleotide, Y represents an antibiotic molecule. When Y represents an oligonucleotide, X represents an antibiotic molecule.

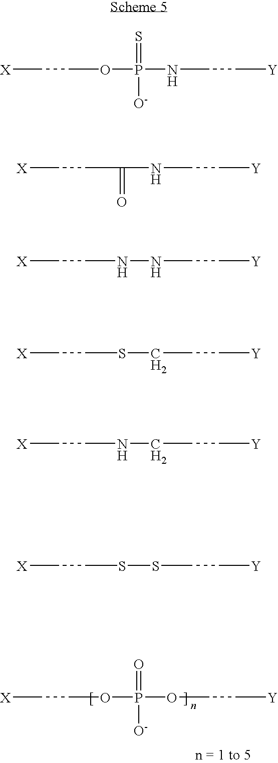

### EXAMPLE 6

### Attachment of Ciprofloxacin to an Oligonucleotide at a Terminal Phosphate

Attachment of ciprofloxacin to an oligonucleotide at a terminal phosphate or thiophosphate is shown in Scheme 6. Ciprofloxacin could attached to the oligonucleotide by formation of a covalent bond between a secondary amine of ciprofloxacin and an activated terminal phosphate of the oligonucleotide.

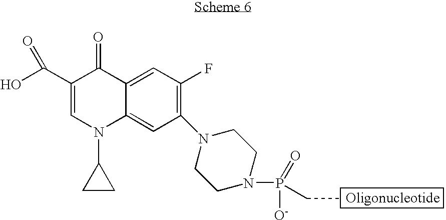

### EXAMPLE 7

### Inhibition of M. Tuberculosis Growth in Broth Culture

M. tuberculosis cultures are set up in duplicates, triplicates, or quadruplicates as 2 ml broth cultures in polystyrene tubes (Fisher, Houston, Tex.) and maintained for 6 weeks. Antibiotic-oligonucleotide conjugates, with oligonucleotides complementary to 30, 32A and 32B genes and prepared according to any of Example 1-6 are added to the medium at a final concentration of 10 μM just before the addition of M. tuberculosis strain Erdman (ATTC 35801; American Type Culture Collection, Manassas, Va.), which is maintained in 7H9 medium (Difco, Detroit, Mich.) supplemented with 2% glucose at 37° C. in a 5% CO₂/95% air atmoshphere as unshaken cultures. Bacterial growth is monitored by gently sonicating the cultures to break up bacterial clumps, removing small aliquots, washing the bacteria by centrifugation, plating serial dilutions of washed bacteria on 7H11 agar (Difco) and enumerating viable bacteria (cfu) after incubation for 2 weeks at 37° C. in a 5% CO₂/95% air atmoshphere. Controls are treated identically, except that the conjugate is replaced by an equimolar amount of antibiotic alone, oligonucleotide separately, or unconjugated antibiotic and oligonucleotide. It is expected that the conjugate will inhibit bacterial growth to a greater extent than any of the controls.

### EXAMPLE 8

### Inhibition of M. Tuberculosis Growth in Human Monocytes

Human acute monocytic leukemia cell line THP-1 (TIB 202; American Type Culture Collection) cells are seeded into wells of Costar (Cambridge, Mass.) 24-well tissue culture plates at a concentration of 5×10⁵ cells per well, differentiated over a period of 36 hours at 37° C. in a 5% CO₂/95% air atmosphere, infected with M. tuberculosis Erdman at a MOI of for 10 in the presence of human serum for 90 minutes, washed, and incubated for 5 days with the antibiotic-oligonucleotide conjugate used in Example 4 at a concentration of 10 μM. The M. tuberculosis bacteria used to infect the THP-1 cultures are first cultured in broth, and then on 7H11 plates for 10 days at 37° C. in a 5% CO₂/95% air atmoshphere before being collected, washed, and diluted to the required concentration. At 3 hours, 2 days, and 5 days after infection, aliquots of the THP-1 cultures are lysed by addition of 0/1% SDS and culture aliquots are serially diluted, plated on 7H11 agar and incubated for 2 weeks at 37° C. in a 5% CO₂/95% air atmoshphere for enumeration of cfu. Cfu from culture aliquots taken 3 hours after infection are used to determine the infection rate and the data point at time “0 days'.

Controls are treated identically, except that the conjugate is replaced by an equimolar amount of antibiotic alone, oligonucleotide separately, or unconjugated antibiotic and oligonucleotide. It is expected that the conjugate will inhibit bacterial growth to a greater extent than any of the controls.

What is claimed is:

1. An antibacterial compound comprising an oligonucleotide having a sequence complementary to a translation initiation region of an mRNA encoding a mycolyl transferase of Mycobacterum tuberculosis selected from protein 30, 32A and 32B, and further having 5′ and 3′ palindromic hairpin-forming sequences, said compound being covalently linked to a protein synthesis inhibiting antibiotic via a linker.

2. The antibacterial compound according to claim 1, wherein the sequence complementary to the translation initiation region is from about 15 to about 50 nucleotides in length.

3. The antibacterial compound according to claim 1, wherein the linker is from 2 to about 50 atomic bond lengths in size.

4. The antibacterial compound according to claim 1 wherein the antibiotic is selected from the group consisting of streptomycin, Sordarin, viomycin, ciprofloxacin, spectinomycin, thiostrepton, virginiamycin M, spramycin I-III, tylosin, carbomycin A, azithromycin, troleandomycin, centhromycin, NRI-A72310, oxazolidinones, lincosamides, sparsomysin, blasticidin S, anisomycin, puromycin, chloramphenicol, tRNA-A76, pulvomycin, enacycloxin IIa, kirromycin, aminoglycosides, tetracycline, glycylcyclines, avilamycin, kasugamycin, pactamycin, edeine and cancer chemotherapeutics.

5. A pharmaceutical formulation comprising an antibacterial compound according to claim 1 and a pharmaceutically acceptable diluent, salt, excipient or carrier.

6. A for treating tuberculosis comprising administering to a patient infected with Mycobacterium tuberculosis a pharmaceutical formulation according to claim 4.


SEQUENCE LISTING (3 sequences)

SEQ ID NO: 1 - DNA, 43 bp, Artificial Sequence
  Description of Artificial Sequence Synthetic oligonucleotide
gcgcatatgcgaatctttcggctcacgtctgtcatgcgcgcgc

SEQ ID NO: 2 - DNA, 43 bp, Artificial Sequence
  Description of Artificial Sequence Synthetic oligonucleotide
gcgcatatgcgacgaaccctgtcaacaagctgcatgcgcgcgc

SEQ ID NO: 3 - DNA, 43 bp, Artificial Sequence
  Description of Artificial Sequence Synthetic oligonucleotide
gcgcatatgcgtcgcacctgttcgaagaacgtcatgcgcgcgc